(19) United States
(12) Patent Application Publication
(10) Pub. No.: US 2020/0090816 A1
(43) Pub. Date: Mar. 19, 2020

(54) Veterinary Professional Animal Tracking and Support System

(71) Applicant: Vet24seven Inc., Mountain View, CA (US)

(72) Inventors: Calbert Lai, Sunnyvale, CA (US); Pamela Seavey, Sunnyvale, CA (US); Edward Blach, Monument, CO (US); Annette Puskarich, Palo Alto, CA (US); Cherice Roth, Boring, OR (US); Tammie Pearce, Tucson, AZ (US); Ross Wright, Austin, TX (US)

(21) Appl. No.: 16/573,622

(22) Filed: Sep. 17, 2019

Related U.S. Application Data
(60) Provisional application No. 62/732,450, filed on Sep. 17, 2018.

(51) Int. Cl. G16H 70/00; G16H 10/60; G16H 30/40; G10L 15/22; G10L 25/78; G10L 15/30

(52) U.S. Cl. CPC G16H 70/00; G16H 10/60; G16H 30/40; G10L 2015/223; G10L 25/78; G10L 15/30; G10L 15/22

(57) ABSTRACT

A system for providing animal related information to animal professionals includes a cloud connected device supporting voice detection and voice response for information receipt and information data input between animal professionals. The voice detection and voice response can be provided by an animal support system that includes a user interface module connected to the cloud connected device to provide non-audio information. Animal related data supplied by the animal support system can be electronically transferrable using a defined format that includes audio information.

### FIELD OF THE INVENTION

This invention relates generally to the field of animal services. A voice controlled virtual assistant usable by veterinary professionals for training or transfer of animal related data and information.

### BACKGROUND OF THE INVENTION

Some of the largest consumer and commercial market segments relate to animal management, feeding, and care, including veterinary services. Pet owners, livestock managers, or zoo personnel can all benefit from knowledgeable selection of food, medicine, or animal related products, timely delivery products or services, and an efficient history, monitoring, or tracking system. When an animal requires medical attention, a simplified system for access to a qualified veterinarian or animal professional for evaluation and treatment is needed.

One particular issue with animal treatment relates to providing veterinarians or other animal support personnel with needed information. Veterinarians are often not able to access animal care relevant data because the veterinarian is actively engaged in handling an animal. This can result in a failure to receive necessary information, or a failure to later record animal related information that is not remembered. Animal treatment can be improved if hands free data provision or recordation is available.

### SUMMARY OF THE INVENTION

In one described embodiment of a method for providing animal related services or products includes receiving an audio request on a cloud connected device supporting voice detection and voice response, the voice detection and voice response is provided by an animal support system. The animal support system can help determine whether at least one of a veterinary consult service and an animal related product is required, with the veterinary consult service being able to provide diagnosis of pet condition based at least in part on a sequence of voiced questions and voiced responses asked and received through the cloud connected device. Selection and payment for needed animal related products can be billed and delivered at least in part using the animal support system. Animal related products can include, but are not limited to, food, pet toys, grooming products, or medicine.

In some embodiments, the cloud connected device is a voice controlled virtual assistant such as may be available in dedicated fixed or portable voice control devices, or used on smartphones, smart watches, tablets, laptop or desktop computers, or the like.

In some embodiments the animal support system has an interactive script for diagnosis of pet condition. In other embodiments, a veterinary consult service can provide access to a person able to answer veterinary related questions. The veterinary consult service can provide access to curated answers, individual animal health records, individual animal genetic sequence records, or other animal breeding records.

In some embodiments, the veterinary consult service can provide access to information derived from grouped animal records, or alternatively, to information derived from animal breed societies.

In some embodiments the veterinary consult service can actively provide information derived from information received through the cloud connected device or alternatively provide information derived from information including animal sounds received by the cloud connected device. In other embodiments the veterinary consult service can actively provide information derived from information including animal images received or relayed from the cloud connected device.

In other described embodiments of a method for providing animal related services to animal professionals, the method includes monitoring audio information using a cloud connected device supporting voice detection and voice response, the voice detection and voice response being provided by an animal support system. Audio request for information receipt and information data input is supported on the cloud connected device, which can also provide audio monitoring of animal condition.

In some embodiments, the cloud connected device is a voice controlled virtual assistant usable by livestock management or workers. Information received via the cloud connected device can include veterinary related questions, curated answers, or access to individual animal health records. The animal support system can actively provide information derived from information received through the cloud connected device, including warnings derived from audio monitoring of animal condition. In some embodiments, the cloud connected device can interact with additional sensors that monitor animal condition, capture animal images, or provide electronically available animal tag identification or sensor information.

In some embodiments, the animal support system can include an ability to respond to voice instructions for food, water, or medicine release, or control of environmental conditions for an animal holding facility such as temperature or lighting.

In another embodiment, a system for providing animal related information to animal professionals includes a cloud connected device supporting voice detection and voice response for information receipt and information data input between animal professionals. The voice detection and voice response can be provided by an animal support system that includes a user interface module connected to the cloud connected device to provide non-audio information. Animal related data supplied by the animal support system can be electronically transferrable using a defined format that includes audio information.

In some embodiments the cloud connected device is a voice controlled virtual assistant usable by veterinary professionals for training or transfer of animal related data and information. The information received can include answers to veterinary related questions, curated answers, individual animal health records. In alternative embodiments, the animal support system can actively provide information derived from information received through the cloud connected device, including information derived from interaction with additional sensors that monitor animal condition. Non-audio information provided by the user interface module can include images, historical sensor data, or even augmented reality video overlay related to an animal.

### BRIEF DESCRIPTION OF THE DRAWINGS

The specific features, aspects and advantages of the present invention will become better understood with regard to the following description and accompanying drawings where:

FIG. 1 illustrates a voice responsive animal care system;

FIG. 2A illustrates use of a voice responsive animal care system to support user interactions with a machine script;

FIG. 2B illustrates use of a voice responsive animal care system to support conversational user interactions that result in receipt of a product or service;

FIG. 2C illustrates use of a voice responsive animal care system to support user interactions that result in a veterinary consult;

FIG. 2D illustrates use of a voice responsive animal care system to support user interactions that result in interaction with other users;

FIG. 2E illustrates use of a voice responsive animal care system to support user interactions;

FIG. 2F illustrates use of a voice responsive animal care system to support user interactions;

FIG. 2G illustrates use of a voice responsive animal care system to support user interactions;

FIG. 2H illustrates use of a voice responsive animal care system to support user interactions;

FIG. 2I illustrates use of a voice responsive animal care system to support user interactions;

FIG. 2J illustrates use of a voice responsive animal care system to support user interactions;

FIG. 2K illustrates use of a voice responsive animal care system to support user interactions;

FIG. 2L illustrates use of a voice responsive animal care system to monitor environmental conditions; and

FIG. 3 illustrates conversational marketing that can be associated with a voice responsive animal care system.

### DETAILED DESCRIPTION

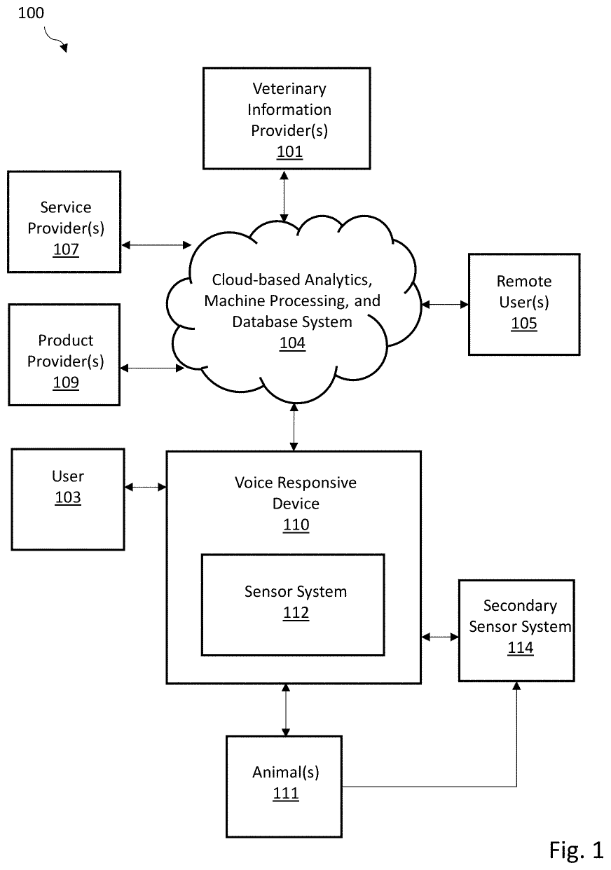

FIG. 1 illustrates a voice responsive animal care system 100 that uses one or more voice responsive devices 110. The voice responsive device 110 can include an on-board sensor system 112 and/or a remotely located secondary sensor system 114. The voice responsive device 110 can be connected to cloud-based analytics, machine processing, and database system 104 that is able to provide various automated services. In addition to the automated services, the cloud-based system 104 can support interactions by a user 103 with other persons or organizations, including veterinary information providers 101, remote users 105, service providers 107, and product providers 109.

The voice responsive device 110 can include custom audio and non-audio sensors, as well as voice identification and recognition hardware/software configured for providing animal related information, services or products. In some embodiments, the voice responsive device 110 can based on commercially available devices. Voice controlled personal assistant systems are widely used as part of fixed or mobile devices, or as an independent application (“app”), as software programs, or as operating system user interface modules for a smartphone, laptop, or other type of computer. Voice recognition, interpretation and response can be done locally, or as part of the cloud-based system 104 that receives filtered and compressed voice or sound recordings, analyzes the recordings, and takes needed actions. Voice synthesis can be used to provide automated spoken replies that can be streamed back to a local device.

In some embodiments, the voice responsive device 110 can include a voice activity detection module to distinguish voice from environmental or animal sounds, a beam forming module to provide a single voice stream derived from multiple microphones, a noise reduction module, a key word detection module, and a command phrase detection module. For example, in situations such as use of system 100 in an animal feedlot, multiple microphones may be needed to adequately cover a noisy area with substantial animal sounds. Voice commands need to be distinguished from animal sounds, environmental noise reduced, and a single voice stream can be used to recognize key words and command phrases. In some embodiments, the voice activity module can be additionally configured to detect a particular animal sound activity (e.g. specific animal distress sounds).

The sensor system 112 includes on-board sensors that are physically adjacent or attached to the voice responsive device 110. The secondary sensor system 114 can be remotely located, but capable of permanent or transient wireless or wired communication with the voice responsive device 110. The sensor system 112 and secondary sensor system 114 can include, but are not limited to, audio microphones, infrasound or ultrasound detectors, air temperature, air pressure, humidity monitors, accelerometers, physical pressure sensors, or position sensors. Video or still camera systems with face recognition capabilities, animal recognition, or other identification, tracking, or object count support can be used. In some embodiments, secondary sensor system 114 can include active animal tag systems that are implantable, attachable to an ear or other animal part, or worn on a collar or harness. These systems can provide a wide range of animal physiological information, including body temperature, pulse rate, oximetry, or animal breathing sounds.

In one aspect, sensor system 112 form a part of mobile devices with video or still camera systems and audio/video (A/V) communication capabilities, such as, for example, smart phones are used to connect animals and veterinarians in ways other than physical proximity. Using a mobile device's camera to take photos and/or video and send those to a veterinarian permits greater degree of detail to be communicated in triaging or diagnosing or follow-up for an animal. Additionally, a camera can be used to conduct a video chat between an animal owner and a veterinarian allowing real-time viewing and conversation between the two parties.

Cloud database 104 can include multiple databases and multiple interfaces. Some interfaces may be optimized for providing response to non-veterinary professional user requests, while other interfaces can be designed for effective use by livestock handlers or veterinary professionals. The cloud database 104 can be capable of storing a wide range of historical service, product, or sensor data (including animal audio related data such as individual sonograms). The cloud database can support manual, semi-automated, or fully automated access. In certain embodiments, standardized formats for veterinary information can be used, simplifying interchange of animal related information between veterinary professionals.

The veterinary information provider can be a local, national, or international business or organization capable of providing a range of animal related information available on a cloud database 104. In addition to factual information about animal and animal health conditions, veterinarian information provider data can include data about the veterinarian or veterinary professional. This information can include type of practice, such as, for example, single or multiple veterinarian, or association with a clinic. Veterinarian data can also include practice name, practice address, practice phone number, indication of single or multiple veterinarian practice. For each veterinarian associated with a practice, veterinarian data can include a name, email, phone number, species treated, hours of availability, academic credentials, specialties (e.g., board certified), a photo, a personal statement, licenses, tax forms, and direct deposit information. Veterinarians can use a veterinarian application to register with the network-based veterinary service provider. Veterinarian registration can include submitting veterinarian data, such as, for example, name, address, phone number, single or multiple veterinarian practice, species treated, credentials, any specialties, licenses, direct deposit information (for receiving payments), clinic information when applicable, a personal statement, etc. Veterinarian data can be stored in a veterinarian database and be made accessible to persons interacting with the voice responsive animal care system 100 or similar network-based veterinary service provider database.

First and second users 103 and 105 are representative, and can include single users, multiple users in a same family, organization, or business, or unrelated users. Users can be in geographically near or remote association. In some embodiments, actual identification and verification of particular users is necessary for access to premium services or to allow for payment for products. Such identification can occur through enrollment in services or systems associated with voice responsive device 110, or can be through separate fingerprint, voiceprint, other biometric services. Password or cryptographic token-based services can also be used. In addition to providing data via a voice responsive device, a user 103 can use a client application to register with the voice responsive animal care system 100 or similar network-based veterinary service provider. Registration can include submitting user 103 data, such as, for example, contact information (e.g., name, mailing address, email, etc.), username, password, billing information (e.g., credit card information), associations with any veterinary clinics, etc. and agreeing to terms of service. User 103 can also setup animal profiles for one or more animals, including animal profile information (e.g., name, age, specifies, breed, genera, photo, etc.). User 103 data, including animal profiles, can be stored in a cloud or user database and be made accessible to persons interacting with the voice responsive animal care system 100 or similar network-based veterinary service provider database.

Service providers 107 can include any animal related service, including but not limited to animal purchase, sale, adoption, feeding, pet sitting, grooming, veterinary services, or animal related information.

Product providers 109 can include any animal related product, including but not limited to animal food, vaccines or medicine, grooming products, or animal toys. Product providers can be online or brick and mortar businesses. Retail, wholesale, or co-op businesses can provide products by direct shipping, mail, parcel delivery services, shipment to a site, by holding for pick-up, or by providing notice of likely or certain store availability.

Animals 111 can be any of a wide range of mammalian, avian, reptilian, amphibian, piscine, or invertebrate animals. Typical pet animals include dogs, cats, horses, rabbits, parrots, snakes, frogs, tropical fish, and tarantulas. Animals typically maintained as livestock can include cattle, pigs, sheep, goats, rabbits, turkeys, or chickens.

Aspects of the system and method can be appreciated with reference to the following exemplary scenarios:

Scenario 1—User Interactions with Machine Sub-System

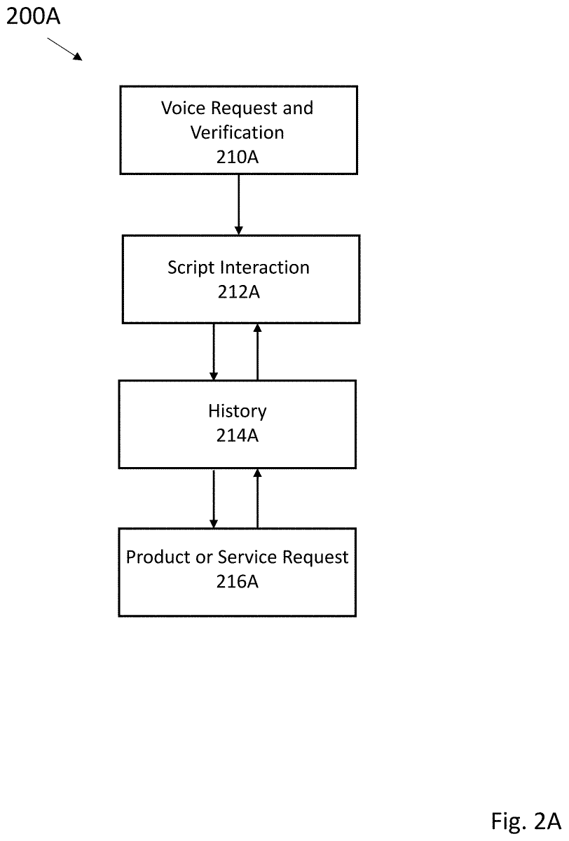

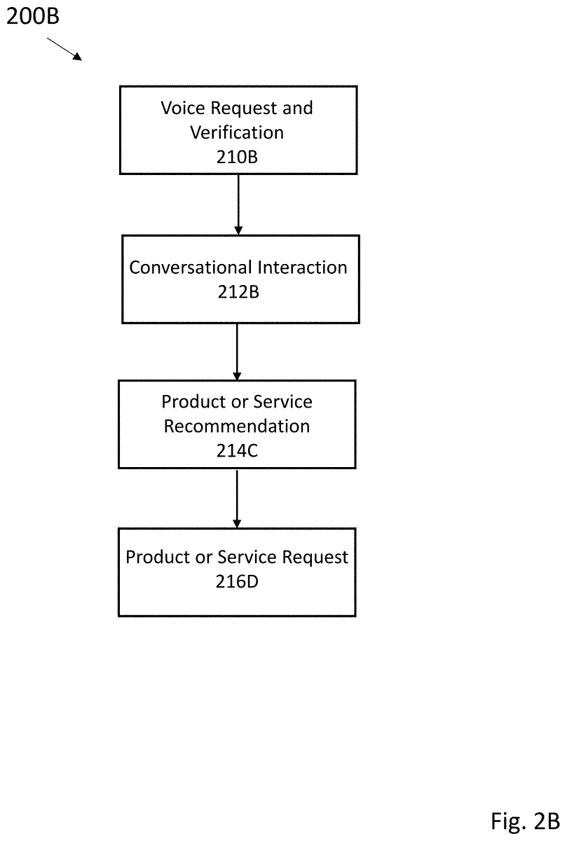

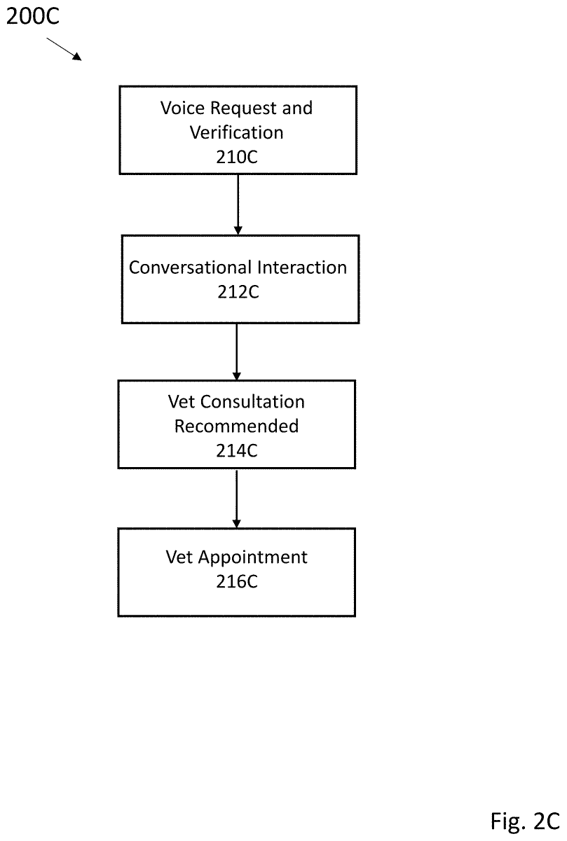

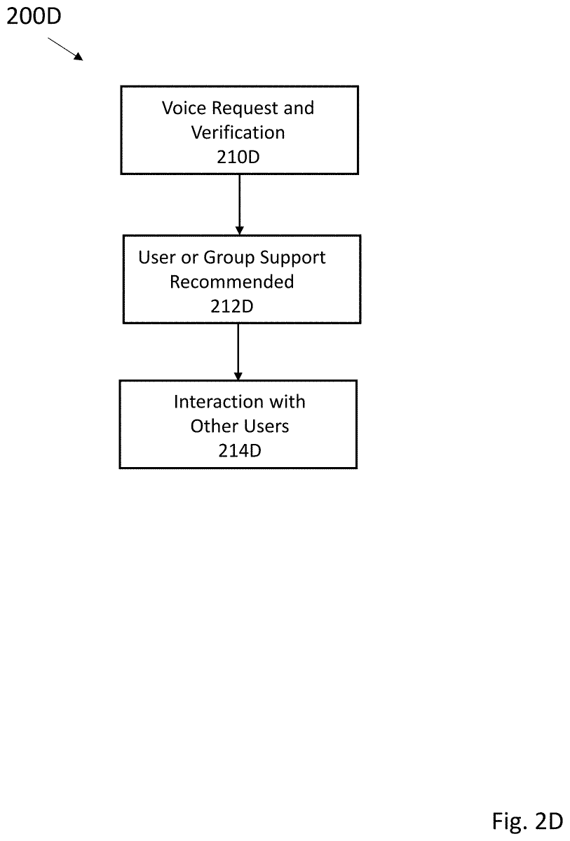

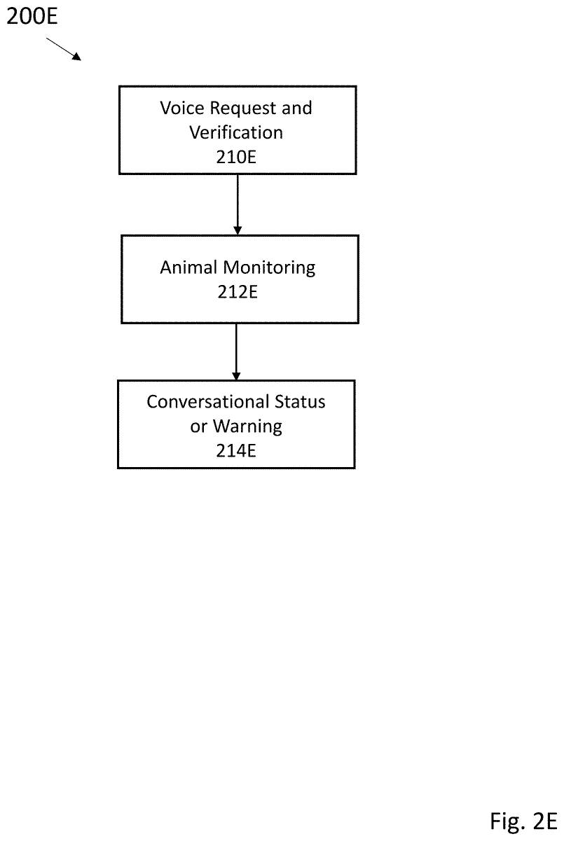

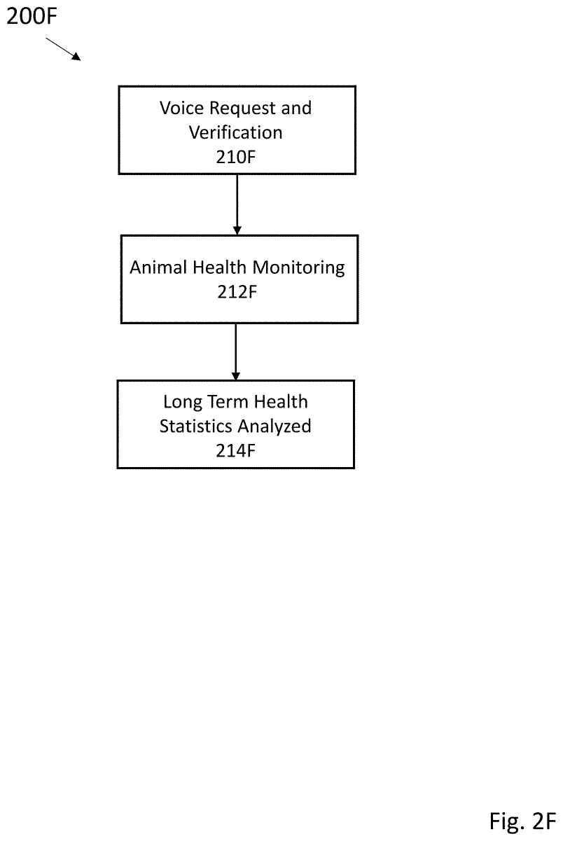

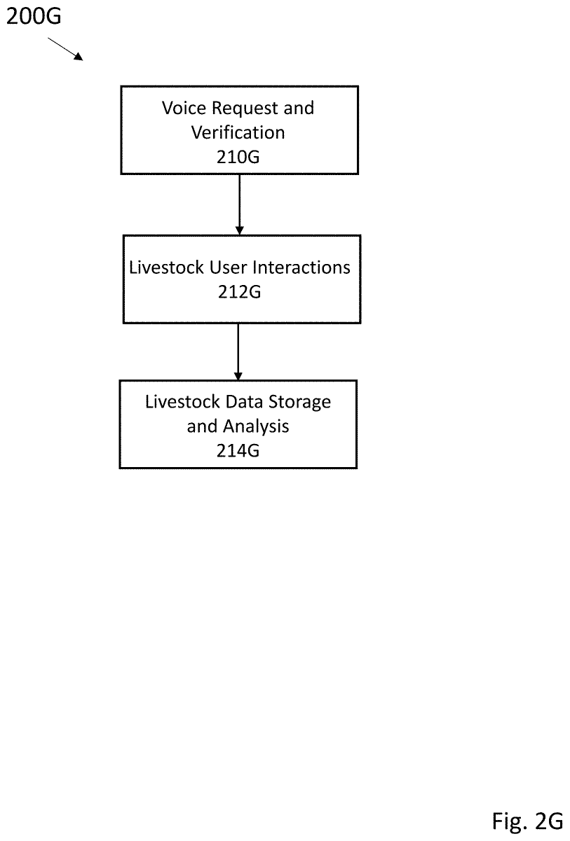

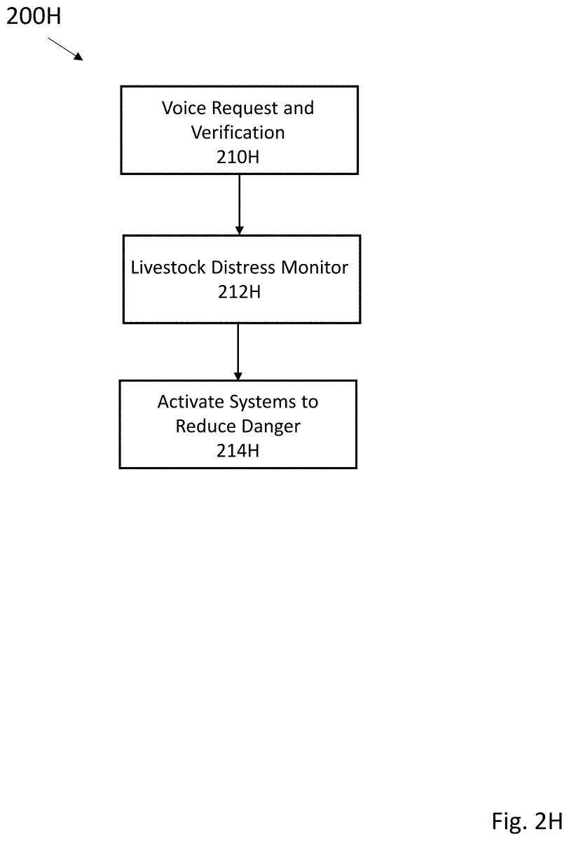

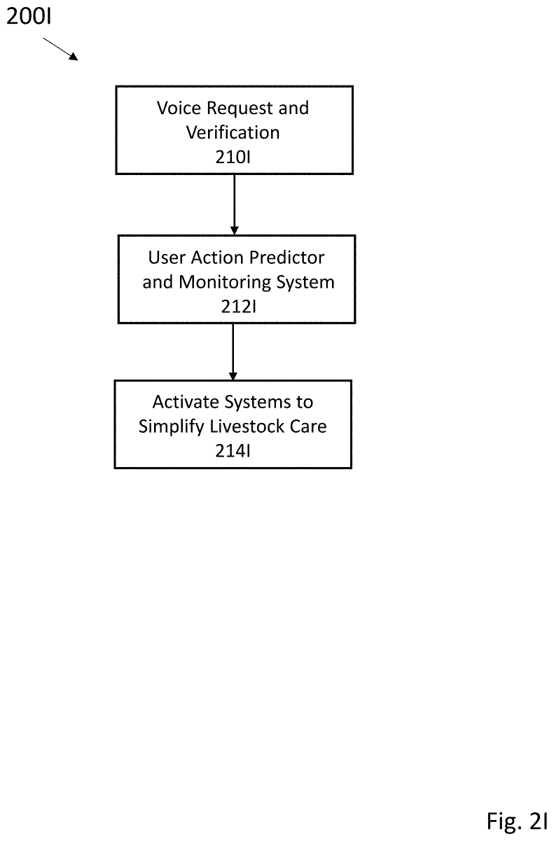

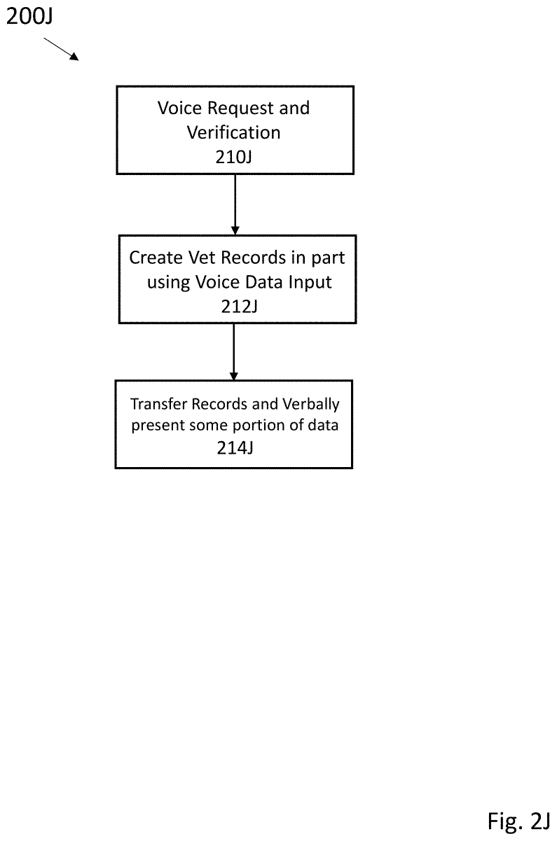

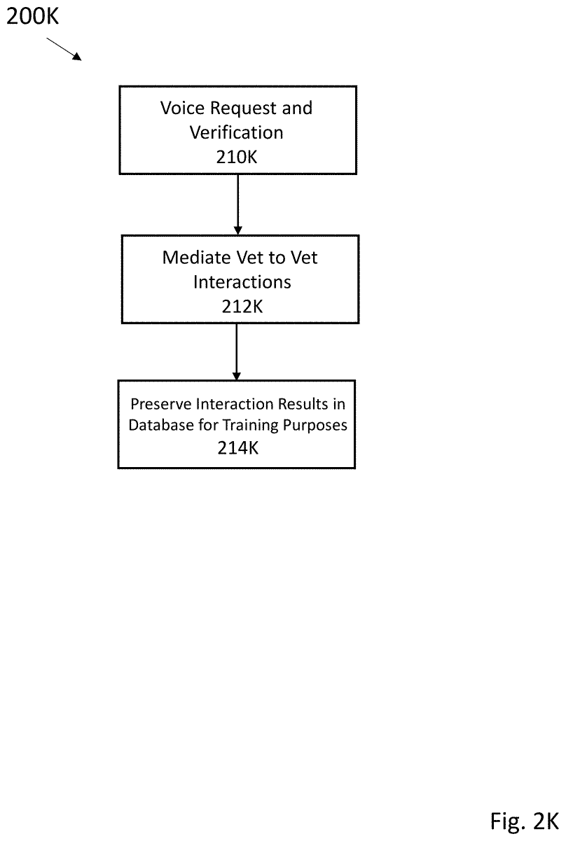

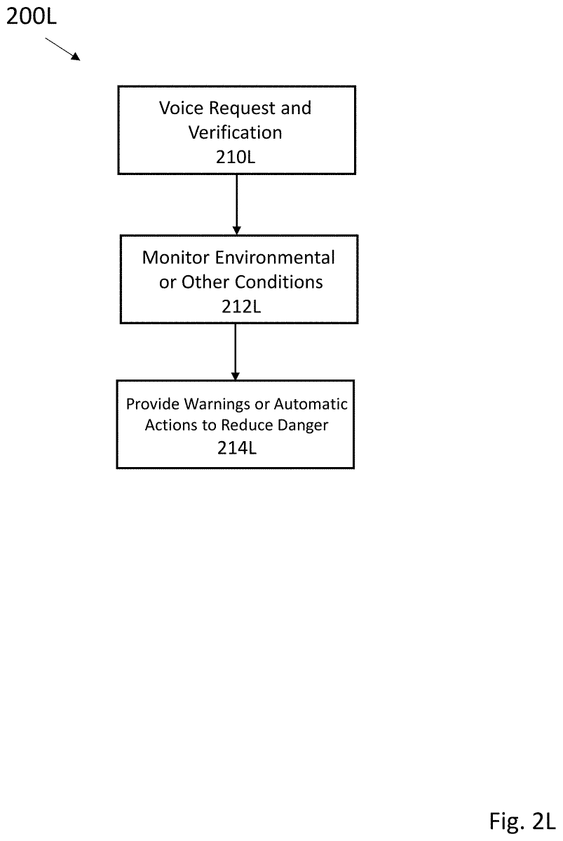

In one embodiment, a user 103 can require advice with respect to behavior or health conditions of an animal. Instead of needing assistance from a live veterinarians or animal care professionals, the system 100 of FIG. 1 can be used. As seen with respect to FIG. 2A, a verified voice request 210A can be made for animal related information. Verification can include but is not limited to voice identity recognition, verification via association with a particular device, biometric identification or voice passwords. In some embodiments, verification is needed before access to certain services or ability to purchase products is allowed.

Following verification, a scripted, expert system, or machine intelligence directed interaction 212A with a cloud-based voice recognition system can occur. Reference can be made to previous scripted interactions and new information can be stored by history module 214A. Based on history or scripted results, recommendations for product purchase or service requests 216A can be made.

Conversational style scripted interactions can be used to provide information with or without a request for services or products. Scripted interactions can be used for animal care emergency triage, or to help direct users to needed products or services.

As an example, a script interaction for feline emergency care can proceed in a manner illustrated as follows:

START

USER: My Cat is vomiting what do I do?

SYSTEM: I am sorry to hear that your cat is vomiting. What is your cat's name?

USER: <Fluffy>

SYSTEM: I need to ask you some questions about <Fluffy>. Is <Fluffy> vomiting blood?

USER: No

SYSTEM: Has <Fluffy> eaten anything like part of a toy, string, fabric, something rubber, plastic or something else that could block the intestinal tract?

USER: No

SYSTEM: Could <Fluffy> have gotten into any human food or human medications?

USER: No

SYSTEM: Has <Fluffy> had any recent changes in diet or treats like flavor, brand or type?

USER: Yes

SYSTEM: It is possible that <Fluffy> may have a sensitivity to something in the new treat or food. Ideally food changes should be made slowly over a 21-day period. If you would like to speak with a veterinarian now to explore this further and to get some helpful hints, I can connect you with a live veterinarian now. Would you like to talk with a veterinarian now? Please answer Yes or No.

USER: Yes

GOTO CLOSE SUBTREE

In an alternative scenario where the answer to the SYSTEM question “Has <Fluffy> had any recent changes in diet or treats like flavor, brand or type?” is “No”, the following alternative script can be used:

SYSTEM: Has <Fluffy> had any recent changes in diet or treats like flavor, brand or type?

USER: Yes

SYSTEM: It is possible that <Fluffy> may have a metabolic issue this refers to a complex of disease processes that are associated with the liver, kidney, pancreas, or the endocrine system (pituitary, thyroid, adrenal glands, ovaries testes). If you would like to speak with a veterinarian now to explore this further and to get some helpful hints, I can connect you with a live veterinarian now. Would you like to talk with a veterinarian now? Please answer Yes or No.

USER: Yes

GOTO CLOSE SUBTREE

Scenario 2—User Interactions with Service or Product Providers

In one embodiment, a user 103 can require provision of grooming or other services and/or products for an animal. The system 100 of FIG. 1 can be used provide such services or goods. As seen with respect to flowchart 200B FIG. 2B, a verified voice request 210B can be made for animal related product or services. Following verification, a conversational interaction 212B with a person, a script, an expert system, or a machine intelligence can occur. Based on history or conversation results, recommendations for product purchase or services 214C can be made. If the recommendation is accepted by the user 103, a product purchase or services request 216D can be made. These can include, but are not limited to products such as food, grooming products, pet toys, medicine or supplements, and services such as pet sitting or walking services, grooming services, and feeding or health monitoring.

Scenario 3—User Interactions with Veterinary Service Providers

In one embodiment, a user 103 can require provision of grooming or other services and/or products for an animal. The system 100 of FIG. 1 can be used provide such services or goods. As seen with respect to flowchart 200C of FIG. 2C, a verified voice request 210C can be made for animal related product or services. Following verification, a conversational interaction 212C with a person, a script, an expert system, or a machine intelligence can occur. Based on history or conversation results, veterinary consultation recommendation 214C can be made. If the recommendation is accepted by the user 103, a veterinary appointment 216C can be made.

In certain embodiments, veterinarian consults requests can be made to veterinarians suitable to handle the veterinary consults. A consult module can receive veterinary consult requests from users of the voice responsive animal care system 100. A veterinary consult request can include consult data defining the parameters of the veterinary consult, such as, for example, a species, urgency, and preferred communication mechanism. Consult module can refer to user 103 data and to veterinarian data for registered veterinarians of the voice responsive animal care system 100 or similar network-based veterinary service provider. Based on the consult data, the user 103 data (including an animal profile), and the veterinarian data for registered veterinarians, the consult module can identify one or more veterinarians suited to handle the veterinary consult request. One of the suitable veterinarians can then accept the request and perform the veterinary consult via the preferred communication mechanism. Once a veterinary consult is accepted, communications between a veterinarian and a user 103 can be established with the same the voice responsive device 110 used to interface with network-based veterinary service provider or alternatively, can be established with different devices.

Typically, a user 103 provides a voice request for a veterinary consult to provide medical analysis with respect to a condition of animal. The user 103 can verbally relates species, animal name, animal symptoms, urgency, and communication type. Species can indicate the species (e.g., dog, cat, bird, etc.) of the animal. Urgency can indicate a time frame for when user 103 desires the veterinary consult to occur, such as, for example, in the next 15 minutes, within 4 hours, sometime today, within a day, etc. Communication type indicates the type of communication preferred by user 103, such as, for example, in-person visits to a veterinarian or animal health professional, in-person visits by a veterinarian or animal health professional, text messages, text chat, audio, audio/video, etc., when conducting the veterinary consult.

In one aspect, user 103 also includes one or more pictures and/or one or more videos of animal representative the animal 111's condition.

In another aspect, user 103 also describes animal 111's condition in one or more text fields. The one or more text fields can include a shorter text field configure to accept 2-3 words describing the animal's condition. The one or more text fields can also include a longer text field configured to accept potentially unlimited text used for a more detailed description of animal 132's condition.

In some embodiments, user 103 data can be provided to and from the cloud database 104. At least one of one or more veterinarians or veterinary professionals can be provided with the veterinary consult request. The consult request can include data provided by that user 103, including but not limited to animal species, the urgency, the preferred communication type, the user data, and any available third party or historical veterinarian data.

In one aspect, prior to notifying the one or more veterinarians a pricing matrix is used to determine a fee for the requested consult. The consult module presents the fee for approval by user 103. If user 103 approves the fee, processing of the consult request continues. Upon approval, the consult module can also pre-authorize a charge to a credit card or other payment mechanism. If user 103 does not approve the fee, processing of the consult request can be terminated. The price presented to a user 103 can be a factor of a pricing matrix, business or operating hours, and method (e.g., live video or chat (messaging). For example, an urgent, live video consult during the day may be priced differently than an urgent, live video consult in the middle of the night.

In one aspect, a veterinarian includes a proposed time to conduct the veterinary consult in consult acceptance. The proposed time can be included in acceptance notification. User 103 can agree to the proposed time or propose a different time. Proposed times can be exchanged until user 103 and veterinarian agree to a time.

During the veterinary consult, additional photos and videos can be transferred form user 103 to the veterinarian or veterinary professional. Prescriptions for animal can be emailed or otherwise communicated to designated recipients including the user 103, a pharmacy, or another person. A summary of the veterinary consult to user 103 and a record of the veterinary consult in added to the file for animal in the cloud database 104.

In one aspect, veterinary consults are conducted via live video or chat (messaging) within the user 103 and veterinarian application. Veterinary consults can be initiated by the veterinarian. When using chat to conduct a veterinary consult, the user 103 and veterinarian are notified when the other party sends a message. To conduct a veterinary consult via video, the veterinarian launches video chat and an invitation to join the video is sent to the user 103 via text message and in-app message. If the user 103 responds to the video invitation, the veterinarian connects with user 103. If the user 103 does not accept the video invitation, the user 103 receives a notification, and the veterinarian can attempt to contact the user 103 again. The video can have an indicator of signal strength and allow sharing of and drawing on photos. More than two individuals can participate in a live video or chat session. The veterinarian and/or user 103 can invite additional participants. For example, a veterinarian can invite another veterinary, for example, a specialist, into a live video or chat session. A user 103 can invite another person interested in the care of the animal, for example, a family member, into a live video or chat session.

In aspects, a user 103 may have a preferred veterinarian. The consult module can direct the user 103′s veterinary consults to the preferred veterinarian when available. When a preferred veterinarian is not available, the consult module can provide the user 103 with alternate veterinarians capable of handling a particular veterinary consult. In a multiple veterinarian practice, alternate veterinarian can be another veterinarian in the same practice. When no other veterinaries are available from the same practice (e.g., when a clinic is closed), the consult module can identify other alternate veterinarians. An alternate veterinarian can be selected based on one or more of: geographic location, licensure, availability, ratings, number of consults performed, species treated, as well as any other information contained in a veterinarian or practice profile.

When the veterinary consult is complete, the payment information for the user 103 is used to bill the user 103 a specified fee. A portion of the specified fee can be retained by the network-based veterinary service provider in exchange for use of a voice responsive user interface platform. The remainder of the specified fee is remitted to the veterinarian, for example, using the direct deposit information.

After a veterinary consult is complete, the user 103 and the veterinarian are provided an option to rate one another.

Scenario 4—User Interactions with Other Remote Users

In one embodiment, a user 103 can interact with other users 105, including animal enthusiasts or appreciation groups, animal advice providers, breeders, or experts on various products and services. The system 100 of FIG. 1 can be used to provide such interactions. As seen with respect to FIG. 2D, a user can make a voice request and receive verification 210D, find (212D) a recommended user or group for support via machine or script recommendation, and interact (214D) with those users. Animal related social interactions can involve contacting skilled non-professionals with an interest in conversation, tips on animal handling, or recommendations or references. For example, a user interested in acquiring a particular animal breed can talk with a remote user and receive advice and recommendations for local breeders.

Scenario 5—Short Term Safety Monitoring of Household Pets

In one embodiment, a user 103 can monitor animal location, sounds, interaction with internet connected toys, food and drink consumption, or animal health status information. The system 100 of FIG. 1 can be used to provide such interactions. As seen with respect to FIG. 2E, a voice responsive device 110 with onboard sensors 112 and optionally associated secondary sensor systems 114 can be used for animal sound monitoring. A user can make a voice request to begin monitoring with various set parameters (e.g. do not monitor when people are in the house) and receive verification (210E), let the voice responsive system monitor animal sounds and other sensed features (212E), and receive conversational warnings with respect to the animals if needed (214E). Monitoring can include individual animal sound and voice recognition. Specific location can be monitored generally, and specifically within a known structure by monitoring the position of an animal-attached device relative to known placements of structure-connected partition transmitters and sensors within a home or other structure, as well as intra-animal interactions (e.g. detected cat fights). Remote detection of animal location, door positions (open or closed), and other features relative to the structure and position of the animal can be controlled or altered remotely.

Scenario 6—Long Term Health Tracking

In one embodiment, a user 103 can allow long term monitoring of animal health status information such as weight, sleep, or activity patterns. The system 100 of FIG. 1 can be used to provide such interactions. As seen with respect to FIG. 2F, a voice responsive device 110 with onboard sensors 112 and optionally associated secondary sensor systems 114 can be used for animal sound monitoring. A user can make a voice request to begin long term health monitoring with various set parameters (e.g. determine animal sleep cycles) and receive verification (210F), let the voice responsive system monitor animal activities and other sensed features (212F), and receive long term health statistics and optional related advice (214E). Monitoring can include individual animal sound recognition relevant to movement, activities, or vocal output that can be used to determine the health, stress levels, and other health parameters of the animal.

Scenario 7—Support for User interactions with Livestock

In one embodiment, a user 103 can be assisted in monitoring status of livestock, determine specific ownership or animal health characteristics, and provide simple livestock related advice, note taking facilities, or other data input capabilities. The system 100 of FIG. 1 can be used to provide such interactions. As seen with respect to FIG. 2G, a voice request with verification can be made (210) Various livestock related user interactions are possible (212G). Caretakers can access triage advice and assistance for helping to categorize and diagnose conditions by submitting symptoms and data via voice or other data generating device. Based upon this triage assistance, appropriate treatment can be recommended as well. Data relevant to these requests can be stored for later analysis (214G), including but limited to determination of the health, stress levels, and other parameters of useful for monitoring livestock.

Scenario 8—Livestock Distress Monitoring

In one embodiment, a remote user 105 can be assisted in monitoring status of livestock, including emergency conditions. The system 100 of FIG. 1 can be used to provide such interactions. As seen with respect to FIG. 2H, animals might be monitored for abnormal noises, movements, or other patterns of behavior that would indicate distress or trouble that managers and caretakers would want to use to signal need to intervene.

Scenario 9—Livestock Environmental Condition Control

In one embodiment, a user 105 can benefit from hands-free interaction with livestock food or drink delivery systems, heating or cooling systems, tag or marker systems, or gates, cages and locks. The system 100 of FIG. 1 can be used to provide such interactions. As seen with respect to FIG. 2I, voice request and verification (210I) can be used in conjunctions with a user action predictor and monitoring system (2121) that is able to activate systems to simplify livestock care (2141). For example, modifiable systems can include environmental systems humidity or temperature control, food availability and delivery systems, or status of cage, gate, or fenced areas. In some embodiments, gates can be selectively opened or closed based on voice request to permit user or animal movement to other spaces.

Scenario 10—Veterinary Data Interchange

In one embodiment, a veterinary information provider 101 can benefit from hands-free provision of animal data via a voice responsive device. The system 100 of FIG. 1 can be used to provide such interactions. As seen with respect to FIG. 2J, after voice request and verification (210J), veterinarian records and other data for patient on-boarding can be accomplished by providing data by voice (212J). Data retrieval from either in-house or third party veterinarian practice management systems can also be accomplished via voice initiation of those actions (214J).

Scenario 11—Veterinary Data Training

In one embodiment, veterinary information for training or inter-professional consultation can be provided to a veterinarian or animal health professional via a voice responsive device. The system 100 of FIG. 1 can be used to provide such interactions. As seen with respect to FIG. 2K, after voice request and verification (210K), veterinarians or other veterinary support can be provided with expert advice in the form of triage guidance, differential diagnosis development, probability calculations and determinations based upon patient and historical data. Access to experts can be live, using voice activation and appropriate mediating devices and systems (212K). Treatment options can be presented via voice and decision assistance provided to optimize medical decision-making. Interaction results can be preserved in a database for training or care tracking purposes ((214K)

Scenario 12—Animal Related Warnings

In one embodiment, users, livestock professionals, or veterinary professionals can be provided with animal related alerts or warnings via a voice responsive device. The system 100 of FIG. 1 can be used to provide such interactions. As seen with respect to FIG. 2L, after voice request and verification (210K), users can be provided with warnings about environmental conditions, changes, or other important criteria that can be monitored, initiated, and altered using this voice technology. Examples include providing rain, wind, or temperature warnings, area pesticide spray warnings, or wild animal warnings.

As will be appreciated various other scenarios are possible and can be implemented on hardware and systems such as described herein.

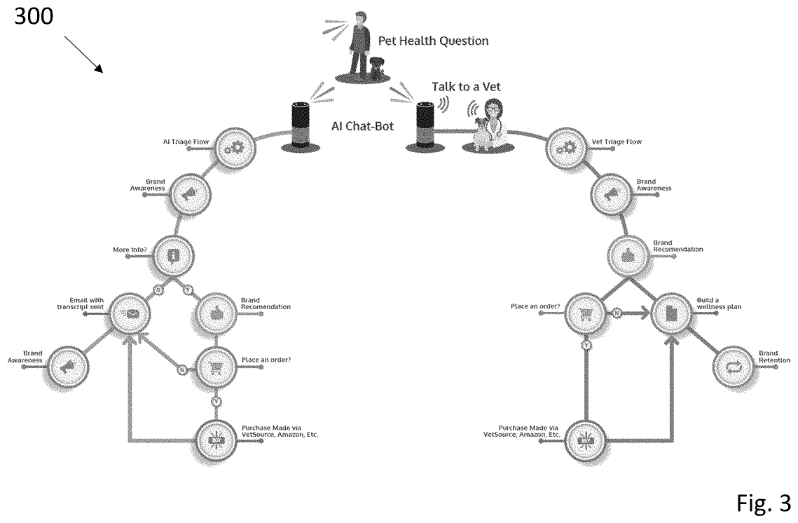

FIG. 3 illustrates one example of conversational marketing that can be associated with a voice responsive animal care system 300. In this embodiment, conversational marketing is can be used to explore differential diagnoses, and upon agreement that the user wants more information on itching and scratching of a dog, for example, and the owner has seen fleas on the dog. In educating about treating fleas, topical and chewable formulations are presented as being most reliable and effective. When the owner states that they prefer chewables, the options of treating every 30 days or every 90 days is presented. Most owners will select to treat every 90 days, and there is only one product currently, Bravecto from Merck Animal Health, that can be administered every 90 days to provide effective protection from fleas. The conversation leads the customer to select chews and every 90 day treatments, so Bravecto is the default selection to meet their needs. This conversation leads them to the only decision that will meet their stated needs.

In the above description of the present invention, reference is made to the accompanying drawings, which form a part hereof, and in which is shown by way of illustration specific embodiments in which the invention is may be practiced. It is understood that other embodiments may be utilized and structural changes may be made without departing from the scope of the present invention.

Embodiments of the present invention may comprise or utilize a special purpose or general-purpose computer including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, embodiments of the invention can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. RAM can also include solid state drives. Thus, it should be understood that computer storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Devices can have touch screens as well as other I/O components.

The described aspects can also be implemented in cloud computing environments. In this description and the following claims, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud computing model can be composed of various characteristics such as, for example, on-demand self-service, broad network access, resource pooling, rapid elasticity, measured service, and so forth. A cloud computing model can also expose various service models, such as, for example, Software as a Service (“SaaS”), Platform as a Service (“PaaS”), and Infrastructure as a Service (“IaaS”). A cloud computing model can also be deployed using different deployment models such as private cloud, community cloud, public cloud, hybrid cloud, and so forth. In this description and in the claims, a “cloud computing environment” is an environment in which cloud computing is employed.

Although the components and modules illustrated herein are shown and described in a particular arrangement, the arrangement of components and modules may be altered to process data in a different manner. In other embodiments, one or more additional components or modules may be added to the described systems, and one or more components or modules may be removed from the described systems. Alternate embodiments may combine two or more of the described components or modules into a single component or module.

The foregoing description has been presented for the purposes of illustration and description. It is not intended to be exhaustive or to limit the invention to the precise form disclosed. Many modifications and variations are possible in light of the above teaching. Further, it should be noted that any or all of the aforementioned alternate embodiments may be used in any combination desired to form additional hybrid embodiments of the invention.

Further, although specific embodiments of the invention have been described and illustrated, the invention is not to be limited to the specific forms or arrangements of parts so described and illustrated. The scope of the invention is to be defined by the claims appended hereto, any future claims submitted here and in different applications, and their equivalents.

What is claimed:

1. A system for providing animal related information to animal professionals, the system comprising:
a cloud connected device supporting voice detection and voice response for information receipt and information data input between animal professionals, the voice detection and voice response provided by an animal support system;
a user interface module connected to the cloud connected device to provide non-audio information; and wherein
animal related data supplied by the animal support system is electronically transferrable using a defined format that includes audio information.

2. The system of claim 1, wherein the cloud connected device is a voice controlled virtual assistant.

3. The system of claim 1, wherein the animal professionals are veterinary professionals.

4. The system of claim 1, wherein information received can include veterinary related questions.

5. The system of claim 1, wherein information received can include curated answers.

6. The system of claim 1, wherein the animal support system can provide access to individual animal health records.

7. The system of claim 1, wherein the animal support system can actively provide information derived from information received through the cloud connected device.

8. The method of claim 1, wherein the cloud connected device can interact with additional sensors monitoring animal condition.

9. The method of claim 1, wherein non-audio information provided by the user interface module can include images.

10. The method of claim 1, wherein non-audio information provided by the user interface module can include historical sensor data.

11. The method of claim 1, wherein non-audio information provided by the user interface module can include augmented reality video overlay.
